(19) United States
(12) Patent Application Publication
(10) Pub. No.: US 2022/0139517 A1
(43) Pub. Date: May 5, 2022

(54) Service for Coordinating Monitoring of REMS Medications

(71) Applicants: Paul Duffy, Alton, NH (US); Glenn Kazo, New Ipswich, NH (US); David Pickar, Chevy Chase, MD (US); James Hunter, Whippany, NJ (US)

(72) Inventors: Paul Duffy, Alton, NH (US); Glenn Kazo, New Ipswich, NH (US); Michael Lusto, Lake Hiawatha, NJ (US); David Pickar, Chevy Chase, MD (US); James Hunter, Whippany, NJ (US)

(21) Appl. No.: 17/464,814

(22) Filed: Sep. 2, 2021

Related U.S. Application Data
(60) Provisional application No. 63/108,381, filed on Nov. 1, 2020.

(51) Int. Cl. G16H 20/10; G16H 40/67; G16H 10/40; G16H 10/60; A61B 5/00; A61K 31/5513; G01N 33/50

(52) U.S. Cl. CPC G16H 20/10; G16H 40/67; G16H 10/40; G01N 33/5094; A61B 5/4839; A61B 5/4082; A61K 31/5513; G16H 10/60

(57) ABSTRACT

A service to coordinate the care of patients who are on a REMS-required medication. The service implements a monitoring hub for coordinating the work between a clinician, pharmacy, and laboratory in the care of a patient. The patient has blood samples tested for safety monitoring. The monitoring hub performs the task of entering the test results . . . into the REMS database. The monitoring hub has qualified personnel who are enrolled in the REMS program as a prescriber designee for the clinician. The prescriber designee works with the clinician, pharmacy, or laboratory to ensure compliance with the REMS requirements. In addition to providing administrative support for REMS compliance, the service could be an integrated care management system that assists patients/families and providers with other aspects of their care and practice needs.

### TECHNICAL FIELD

This invention relates to the monitoring of patients on medications having serious side effects.

### BACKGROUND

Patients with Parkinson disease often develop further mental conditions as brain neurodegeneration progresses, such as psychosis (hallucinations and delusions). These secondary neurologic conditions are often treated with antipsychotic medications such as clozapine. However, clozapine can cause serious, life-threatening, adverse effects. One of the most worrisome adverse effects of clozapine is its propensity to induce loss of neutrophils in the blood (i.e. neutropenia). If the drug causes the blood absolute neutrophil count (ANC) to drop below 500/μL, this is considered severe neutropenia and requires immediate discontinuation of the drug.

To better manage the risks associated with clozapine, the Food & Drug Administration (FDA) implemented a mandatory program called the Clozapine Risk Evaluation and Mitigation Strategy (REMS). This REMS program is a patient registry that is shared among the various healthcare workers, including the clinician and the pharmacy. The REMS program may be implemented as an online portal where users login within a username and password. The online portal at {www.clozapinerems.com} is an example.

Patients who are receiving clozapine are enrolled in the registry. This REMS program mitigates the risk of neutropenia by educating prescribers and pharmacists about the risk of neutropenia and appropriate monitoring requirements, informing patients about the risk of severe neutropenia and appropriate monitoring requirements, promoting compliance with blood test monitoring for ANC prior to dispensing clozapine, and encouraging the prescriber makes a risk-benefit assessment when the patient's ANC falls below the acceptable range.

The current workflow for REMS clozapine monitoring is shown in FIG. 1. Both the clinician 56 and the pharmacy 58 are registered in the clozapine REMS program 90. To become certified and registered, clinician 56 completes the necessary forms and mails or faxes them to the REMS program 90. In step 1, clinician 56 prescribes clozapine for a patient 52 with Parkinson disease. In step 2, clinician 56 sends this prescription information to the pharmacy 58. In step 3, clinician 56 also issues a standing order for blood draw samples to be collected from the patient 52 at regular intervals (e.g. weekly, biweekly, monthly, etc.) and analyzed by a laboratory 60 for absolute neutrophil count (ANC). The blood draw collection may be performed at a clinic or at the laboratory 60 site. Although pharmacy 58 has received the clozapine prescription, it does not dispense the medication until the REMS program 90 confirms that the patient's 52 ANC is within safe limits and gives authorization. Thus, in step 4, the patient 52 visits the clinic or laboratory 60 for blood sample collection. The laboratory 60 analyzes the blood sample for ANC. In step 5, the test results are transmitted to the clinician 56. In step 6, clinician 56 enters the ANC test result into the database of REMS program 90. In step 7, if the ANC is within acceptable range, the REMS program 90 gives authorization to pharmacy 58 to dispense the medication to the patient 52. The patient 52 is contacted that the medication is ready for pick-up. In step 8, the patient 52 then visits pharmacy 58 to pick-up the clozapine medication.

As seen here, this is a complex workflow that requires coordination at multiple points and back-and-forth transmission of information between multiple parties. There are numerous gaps where breakdown or delay in communication flow could occur and jeopardize patient safety. Thus, there is a need for a more modernized system for coordinating these administrative tasks relating to REMS compliance and reducing the administrative burden on the clinician.

### SUMMARY

This invention provides a service to coordinate the care of patients who are on a REMS-required medication. As used herein, the term “REMS-required medication” means a medication that is required to be administered under the FDA's Risk Evaluation and Mitigation Strategy (REMS) program. This invention could be used for any of the REMS-required medications. A list of such REMS-required medications are available at {www.accessdata.fda.gov/scripts/cder/rems/index.cfm}, which is incorporated by reference herein. As used herein, “REMS program” means the Food & Drug Administration mandated ‘Risk Evaluation and Mitigation Strategy’ drug safety program for certain medications. The REMS program includes the collection of elements that work to administer the requirements, such as personnel, set of instructions or standards, computer systems and software, algorithms, databases, online interfaces, network equipment, etc.

The invention implements a monitoring hub for coordinating the work between a clinician, pharmacy, and laboratory in the care of a patient. The patient may reside in any setting, such personal home, independent living community, residential care facility, nursing facility, etc. In some embodiments, the patient has Parkinson disease and a secondary neurologic condition (such as psychosis) that is treatable with a REMS-required medication. In some embodiments, the REMS-required medication is an antipsychotic medication such as clozapine, olanzapine (Zyprexa), or loxapine (Adasuve). In the particular case of clozapine, the side effect of neutropenia (low blood neutrophil count) may be of concern.

As used herein, the term “clinician” means a physician, nurse practitioner, physician assistant, or any other healthcare practitioner with authority to prescribe medications. In this invention, the clinician is certified in the REMS program for the particular medication. As used herein, the term “transmit” means communicating information from one party to another party by any suitable means, including verbal (e.g. phone call), fax, email, text message, video-conference, or other electronic communication system. In any instance where one party transmits information, the other party is inferred to ‘receive’ the information. And vice versa, in any instance where one party receives information, the other party is inferred to transmit the information.

As used herein, the term “monitoring hub” means a collection of one or more sites that are physically located remotely from the clinician site, the pharmacy site, and the laboratory site. The “monitoring hub” includes the personnel along with the computer systems, communication systems, and other equipment to perform the work. The monitoring hub may be located at a single physical site, or alternatively, there may be multiple (two or more) physical sites that collectively constitute the monitoring hub (e.g. distributed team or workforce). For example, there may be a single main physical site, and remote workers from home, that collectively constitute the monitoring hub.

In one aspect, the invention is a method of managing the care of a patient who is on a REMS-required medication. The method comprises the following steps that are already practiced in the prior art. The clinician makes a treatment plan for the patient. This treatment plan comprises (i) a prescription for a REMS-required medication for the patient; and (ii) a safety monitoring plan for blood testing from collection of blood samples taken from the patient at intermittent times. The safety monitoring plan includes relevant medical orders for collection of blood samples from the patient and laboratory analysis thereof.

The prescription is transmitted to a pharmacy. In some embodiments, the pharmacy is an outpatient pharmacy, such as retail drugstore pharmacies, specialty pharmacies, ambulatory care pharmacies, pharmacies dispensing to long-term or rehabilitation facilities, etc. A blood sample is taken from the patient and analyzed at a clinical laboratory. This results in the generation of blood test results, which includes the collection date of the blood sample. The blood test results are reviewed for acceptable parameters. Based on the blood test results, the pharmacy dispenses the medication to the patient. As the patient continues use of the medication, repeat blood samples are taken from the patient. Based on review of the repeat blood test results, the pharmacy continues dispensing the medication to the patient.

Improvement: The improvement in this method involves the role of a monitoring hub to coordinate the work process. The improved method comprises one or more of the following steps. The monitoring hub has one or more personnel who are enrolled in the REMS program as a prescriber designee for the clinician. The patient's prescription information is transmitted to the monitoring hub (e.g. by the clinician or the pharmacy). Information about the safety monitoring plan is also transmitted to the monitoring hub (e.g. by the clinician or the laboratory).

The blood draw procedure may be performed at any suitable facility, including medical centers, clinical offices, specialized patient facilities, or at the laboratory site. Alternatively, the blood draw may be performed at the patient's residence by a visiting patient worker, as described in more detail below. The monitoring hub receives the blood test results (e.g. from the clinician or directly from the laboratory). The monitoring hub enters the blood test results into the REMS database.

The prescriber designee personnel at the monitoring hub reviews the blood test results and confirms for acceptable parameters (e.g. acceptable test values and collection date) as given in the predefined safety criteria. Optionally, the monitoring hub may then transmit the confirmation to the pharmacy. If a blood test result is not within acceptable parameter(s), the prescriber designee requests instructions from the clinician about changes to the treatment plan.

Other Optional Improvements: There are numerous other possible features and other possible pathways that could be part of the monitoring hub service to improve coordination of patient care. Among them are one or more of the following. The monitoring hub may perform the task of enrolling the patient into the REMS program. The clinician may transmit the prescription to the monitoring hub, and then the monitoring hub may transmit the prescription to the pharmacy. The clinician may transmit the safety monitoring plan to the monitoring hub, and then the monitoring hub may transmit the safety monitoring plan to the laboratory. The monitoring hub may arrange for the blood collection to be performed (e.g. scheduling, workers, transportation, etc.). The monitoring hub may receive the blood test results from the laboratory, and then transmit those blood test results to the pharmacy or clinician. Some of such tasks performed by the monitoring hub may be redundant to those already performed by the clinician, pharmacy, or laboratory.

The prescriber designee personnel may also review the patient's prior blood test results (e.g. ANC history). In some cases, the prescriber designee personnel changes the safety monitoring plan (e.g. change the blood testing frequency) based on reviewing the history of blood test results according to pre-established REMS guidelines. For example, for clozapine in a general population patient, the patient may start with weekly testing at initiation, and with continued acceptable ANC results for 6 months duration, reduced to biweekly testing, and with further continued acceptable ANC results for another 6 months duration, reduced further to monthly testing. This is according to established guidelines, which are available from various sources such as {www.clozapinerems.com}, which is incorporated by reference herein.

In some embodiments, the monitoring hub has one or more visiting patient workers. A visiting patient worker could visit the patient at their residence to perform the blood collection. The visiting patient worker could be a licensed nurse, phlebotomist technician, nurse assistant, or other healthcare worker able to perform the relevant duties. The visiting patient worker could help the patient with various other medical-related needs, such as transporting the blood sample to the laboratory, arranging patient travel logistics (e.g. arranging for transportation to the laboratory, pharmacy, or clinician), ensure that the patient is taking their medications correctly, answering questions and addressing issues raised by the patient or family, and helping with the technology provided by the monitoring hub (such as videoconferencing). The visiting patient worker could also alert the clinician to any deterioration in patient status.

Monitoring System: In another aspect, the invention is a system for monitoring a patient on a REMS-required medication. The system comprises: (i) a clinical practice comprising a clinician; (ii) a pharmacy comprising a pharmacist; and (iii) a clinical laboratory. The clinician, pharmacy, and clinical laboratory each perform the tasks as already described above. In the system of this invention, the improvement comprises (iv) a monitoring hub that has personnel who is enrolled in the REMS program as a prescriber designee on behalf of the clinician. The monitoring hub receives from the clinician, patient information about the patient and the treatment plan for the patient. The monitoring hub also receives the blood test results from the laboratory and enters the blood test results into the REMS database. The prescriber designee reviews the blood test results for acceptable parameter(s). If a blood test result is not within acceptable parameter(s), the prescriber designee requests instructions from the clinician about changes to the treatment plan. The system may further have other components or functions as already described above.

### BRIEF DESCRIPTION OF THE DRAWINGS

FIG. 1 shows an example how the REMS clozapine monitoring is currently performed.

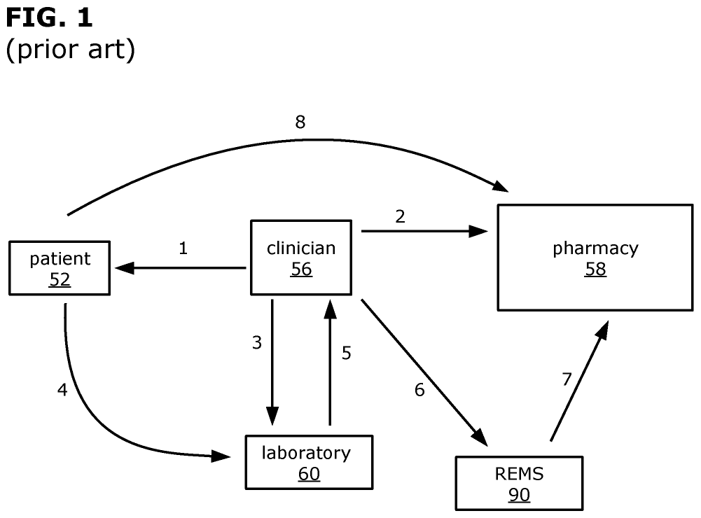

FIG. 2 shows an example of how REMS clozapine monitoring could be facilitated by a monitoring hub.

FIG. 3 shows an example of how the monitoring hub could be part of an integrated care and patient safety management system.

FIG. 4 shows a block diagram of a computer system with software installed thereon that could be implemented by the monitoring hub.

### DETAILED DESCRIPTION OF EXAMPLE EMBODIMENTS

To assist in understanding the invention, reference is made to the accompanying drawings to show by way of illustration specific embodiments in which the invention may be practiced. The drawings herein are not necessarily made to scale or actual proportions. For example, lengths and widths of the components may be adjusted to accommodate the page size.

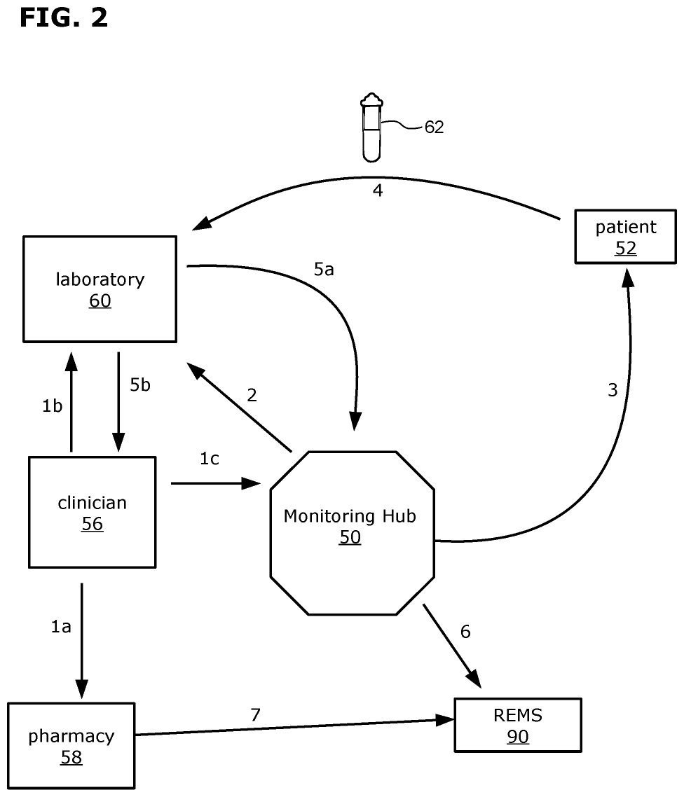

FIG. 2 shows a block diagram of the workflow that can be facilitated by a monitoring hub 50. The patient 52 in this scenario has Parkinson's disease and resides at the patient's home or in an independent living assisted care home. The patient 52 is dependent on family, friends, or care assistants for tasks such as daily activities, transportation, meals, and administration of medications. The prescribing clinician 56 assesses the patient 52 and decides that clozapine would be beneficial for the patient 52. The prescribing clinician 56 sends the patient information and details about the clozapine treatment plan (e.g. dosage amount) to monitoring hub 50, and requests that the patient 52 be enrolled into the REMS program 90. The hub 50 performs the task of enrolling the patient 52 into the REMS program 90.

If clinician 56 is not already certified in the REMS program 90, monitoring hub 50 could also perform the work in certifying or enrolling clinician 56 into the REMS program 90. For example, monitoring hub 50 could provide clinician 56 with the required educational information about clozapine and its risks, and also complete the ‘clozapine REMS prescriber enrollment form’ for clinician 56.

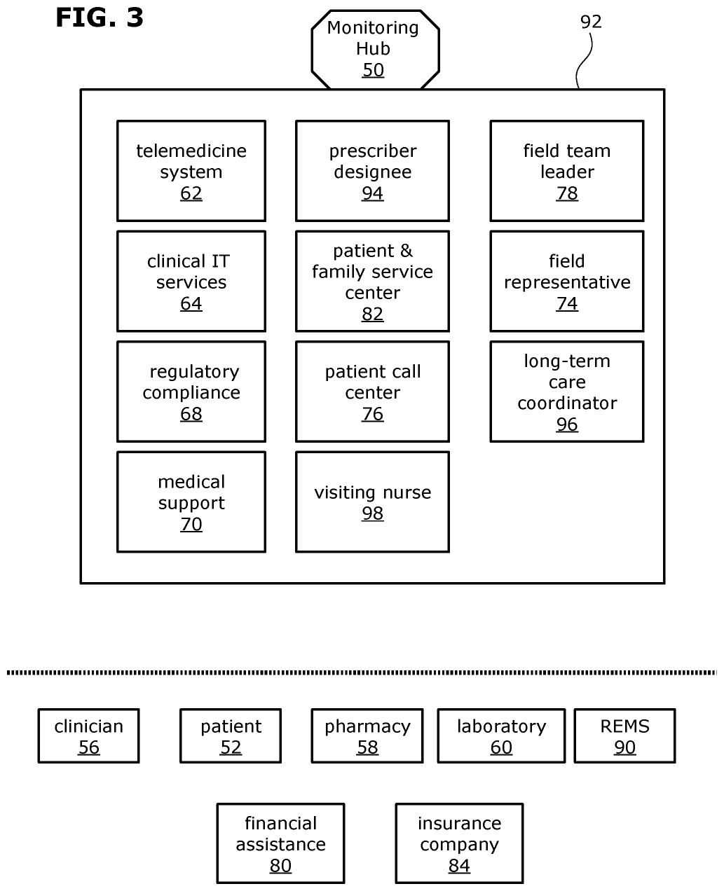

The monitoring hub 50 has one or more personnel who are qualified to serve as a ‘prescriber designee’ 94 (see FIG. 3) on behalf of prescribing clinician 56. For example, such personnel could be another clinician, nurse, or a pharmacist. The prescribing clinician 56 designates such personnel to be a prescriber designee 94 on their behalf. After receiving the patient information and treatment information, monitoring hub 50 enrolls the patient 52 into the REMS program 90.

In step 1a, the prescribing clinician 56 transmits a prescription for clozapine to the pharmacy 58 to be dispensed for the patient 52. In step 1b, clinician issues a standing order for blood draw samples to be collected from the patient 52 at regular intervals (e.g. weekly, biweekly, monthly, etc.) and analyzed by a laboratory 60 for absolute neutrophil count (ANC). In step 1c, clinician 56 also transmits this prescription information and blood testing order to the monitoring hub 50.

In step 2, monitoring hub 50 contacts laboratory 60 to inform them that monitoring hub 50 will arrange for a blood draw to be taken from the patient 52 at the patient's home or independent living assisted care home. Prior to initiating clozapine treatment, the patient's 52 baseline ANC must be established. The monitoring hub 50 has a licensed nurse 98 (see FIG. 3) who is capable of performing blood draws. In step 3, the monitoring hub's 50 nurse 98 travels to the patient's home or independent living assisted care home and performs a blood draw on the patient 52. In step 4, the nurse 98 transports the blood draw sample 62 to the laboratory 60, which performs an absolute neutrophil count (ANC) on blood sample 62. Alternatively, nurse 98 could have point-of-care testing equipment that allows blood testing on-site. In this alternate scenario, nurse 98 would perform the point-of-care testing on blood draw sample 62 and electronically transmit the results to laboratory 60 and monitoring hub 50. In steps 5a and 5b, the laboratory results and the date of blood collection are sent to both prescribing clinician 56 and monitoring hub 50.

Monitoring hub 50 confirms that the blood collection date is within an acceptable date range based on the patient's 52 monitoring frequency to ensure that proper authorization for dispensing clozapine is made. In step 6, monitoring hub 50 enters the ANC test result into the database of REMS program 90. In step 7, pharmacy 58 logs into the REMS program 90 and confirms that the patient's 52 ANC is within an acceptable range and of sufficiently recent collection date. With this, the REMS program 90 gives authorization to pharmacy 58 to dispense clozapine to patient 52.

For the first dispensing, the REMS program 90 could verify that pharmacy 58 is certified, that patient 52 is enrolled, and that patient's 52 treatment is not interrupted or discontinued. For subsequent dispensing, the REMS program 90 could verify that pharmacy 58 is certified, that patient 52 is enrolled, that a ‘patient status form’ has been completed in the last 37 days, and that patient's 52 treatment is not interrupted or discontinued. Pharmacy 58 fills the clozapine medication and has it ready for pick-up. A pharmacy transportation service picks-up the clozapine and delivers it to the nursing home for administration to the patient 52.

The monitoring hub's 50 nurse 98 continues to visit patient 52 to perform blood draws according to the schedule set by clinician's 56 standing order for blood draw samples. The follow-up laboratory results continue to be transmitted to monitoring hub 50, where the follow-up results are entered into the database of REMS program 90 electronically. The prescriber designee 94 at monitoring hub 50 reviews the laboratory results and confirms that the patient's 52 ANC≥1500/μL. The pharmacy 58 sees the acceptable ANC level in the REMS database and continues to dispense the clozapine medication to patient 52 accordingly.

If the patient's 52 ANC is lower than the acceptable range, then the prescriber designee 94 informs clinician 56 about the situation. After evaluating the situation, clinician 56 informs the prescriber designee 94 about any changes to the treatment plan, such as interrupting the clozapine treatment, reducing the dosage, repeating the ANC test, performing ANC testing more frequently, etc. The prescriber designee 94 enters any new treatment plan information into the database of the REMS program 90. The monitoring hub 50 also sends the nurse 98 to visit the patient 52 to inform the patient 52, family members, or nursing home staff about the changes and answer any questions. Prescriber designee 94 could also instruct visiting nurse 98 to arrange a videoconference as a virtual clinic visit with the patient 52 and family members. This videoconference could be with the prescriber designee 94 or clinician 56 (i.e. a virtual doctor visit).

The prescriber designee 94 at monitoring hub 50 could also revise the blood draw frequency for patient 52 according to the standard protocol. For example, the ANC monitoring frequency may be reduced from weekly to biweekly if ANC remains stable for a duration of 6 months, and then reduced further to monthly if the ANC continues to remain stable for a duration of another 6 months.

In compliance with FDA regulations, the hub 50 submits a ‘patient status form’ to the REMS program 90 on a monthly basis. The ‘patient status form’ that records the patient 52 ANC. Under FDA regulations, the ‘patient status form’ may be used to: *interrupt, discontinue, or resume treatment; *designate the patient as a ‘benign ethnic neutropenia’ (BEN) patient; *create a treatment rationale when the patient's ANC level is <1000/μL for a general population patient or <500/μL for a BEN patient; *designate the patient as a hospice patient.

In a scenario different from that of FIG. 2, if the patient 52 resides in a nursing home with professional nursing care, the nursing home would have its own staff of nurses and clinicians. In this situation, a long-term care coordinator 96 (see below, FIG. 3) could acting as a liaison between patients and the care facility, ensure that the care facility staff are educated about the medication and its side effects, and ensure that the patient meets all institutional criteria. More details about the role of long-term care coordinator 96 are given below.

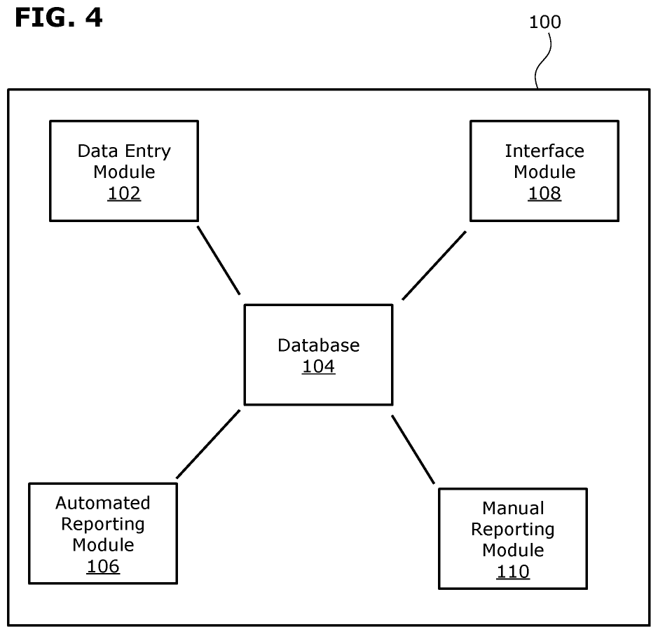

FIG. 4 shows a block diagram of a computer system 100 with software installed thereon that could be implemented by the monitoring hub 50. The software loaded on computer system 100 comprises a data entry module 102, database 104, interface module 108, automated reporting module 106, and manual reporting module 110. Data entry module 102 provides user interfaces for entry of data into database 104. Data entry module 102 is capable of performing a variety of different types of functions or data transactions. For new patient data, it could be coded to contain specific fields required for entering new patients and prescriptions into the database 104 for the first time. For existing patients, it could be coded to contain specific fields required for adding, removing, or editing data for patients who are already entered into the database 104. For making location updates, it could be coded to contain fields specific to geographic locations to include site, city, state, and zip code to allow patient locations to be updated as they move their residence. Data entry module 102 could also be coded to contain fields specific for prior prescriptions or prior patient activities. Data entry module 102 could also allow searching by specific fields such as location, social security number, patient ID, or patient name.

Database 104 may contain various types of electronic files that contain information relevant to implementing the REMS monitoring service. For example, such files may include data about the patients (age, gender, family, etc.), medications and treatment plan, blood test monitoring plan, clinicians and other healthcare workers (location, REMS certification information, associated patients, etc.), pharmacies (location, REMS certification information, associated patients, etc.), medical insurance or coverage, laboratory data, radiological or imaging data, etc.

The interface module 108 gives the system the ability to interface with other organizations' systems for exchanging data (e.g. receiving or transmitting). For example, computer system 100 could communicate and exchange data with the database of the REMS program 90 (see FIGS. 2 and 3 above), or with the computer systems used by pharmacy 58, clinician 56, or laboratory 60. Interface module 108 could also have data export capabilities so that users can export data for use by other software tools.

The automated reporting module 106 generates patient reports at regular intervals (e.g. weekly reports). For example, these may be pre-designed automatically generated reports about each patient being monitored by hub 50. These reports may give a summary of the patient's test results, blood monitoring frequency requirements, next scheduled blood draw, comments from the prescriber designee 94, medication quantity and dosage dispensed by pharmacy 58, comments from visiting nurse 98, and timeliness of blood draw collections, and next scheduled visit with clinician 56. These reports may be automatically sent to other parties, such as pharmacy 58, clinician 56, or laboratory 60. The manual reporting module 110 provides a list of searchable fields in which the user can create reports as the need arises.

Additionally, although this particular computer system 100 is illustrated as a single site, the components of computer system 100 and its associated software could also be designed as a distributed system. Thus, for instance, several computers may be in communication by way of a network connection and may collectively perform tasks described as being performed by the computer system 100. As described here, another embodiment of this invention is a software product that executes the functions of the described computer system 100 (i.e. a non-transitory computer-readable storage medium storing instructions that when executed by a computer system, causes the computer system to perform the recited steps of the service method or functions of the computer system). In addition to providing administrative support for REMS compliance, this invention could be an integrated care management system that assists patients and providers with other aspects of their care and practice needs. These may be particularly useful for elderly and frail patients who are home-bound without reliable transportation or family support. FIG. 3 shows another example of this invention with additional features for monitoring hub 50 to form an integrated care management system 92 for the various parties involved. The integrated care management system 92 may engage with various third parties to improve coordination of care to the patient 52 as well as enhance operational effectiveness and efficiency for clinician 56 or pharmacy 58.

Insurance Companies: Care management system 92 could interact with insurance companies or Medicaid/Medicare to ensure that clinician 56 will receive proper reimbursement for billings. Care management system 92 may assist in obtaining insurance authorization by contacting the relevant insurance company 84 to obtain authorization for the charges associated with the services. These charges could be for the laboratory charges, visiting nurse charges, medication charges, clinician charges, etc. Care management system 92 may request pre-authorization for the services provided by care management system 92. Medicare payment procedures could be electronically coded to provide for payment directly to the caregivers/vendors.

Patient Call Center: Care management system 92 could include a patient call center 76 to receive inquiries or remarks from patients, family, or their care assistants. Patient call center 76 may receive the calls by any conventional means, such as telephone, video, text messaging, email, etc. Patient call center 76 may process patient complaints, receive requests for medical consultation, setup a follow-up appointment to the relevant clinician, or place the patient in contact with an ‘on-call’ clinician to assist with any urgent problems.

Regulatory Compliance: Care management system 92 could have a regulatory compliance division 68 to assist clinician 56 or the clinical practice with various regulatory compliance tasks. Such tasks can include instituting a mechanism for compliance with patient privacy rules, staff training, proper billing for Medicare/Medicaid services, review of independent contractor arrangements, performing regular audits of compliance practices, performing or coordinating investigations of deficiencies, initiating or coordinating corrective and preventive action for areas of noncompliance, etc.

Clinical IT Services: Care management system 92 could have a clinical IT services division 64 to assist with the information technology requirements of clinician 56 or the clinical practice. Care management system 92 could assist the clinical practice with various products or services to improve clinical practice operations, such as automation of medical billing and collections, electronic practice management programs, electronic health record systems, systems integration with other third-party systems, etc.

Telemedicine System: Care management system 92 could also provide a telemedicine system 62 to clinician 56 or the clinical practice to allow remote communications with patients. Telemedicine system 62 may employ any suitable electronic communication technology, such as phone calls, video chats, emails, or text messages. Telemedicine system 62 may have various features that are conventionally included in such systems. For example, telemedicine system 62 may have a patient portal to send/receive messages from clinician 56 or clinician's staff, request prescription refills, setup appointments, give access to laboratory or imaging test results, etc. Telemedicine system 62 may also be capable of implementing virtual appointments. For example, patient consultations can be performed by phone call or video conference.

Medical Science Liaison (MSL) Support: Care management system 92 could also provide medical science liaisons, such as a field team leader 78 to clinician 56 or the clinical practice, or field representative 74 for pharmacy 58. Such medical science liaison support 74,78 are trained to provide technical assistance with the technical aspects of the care management system 92, give the latest medical or scientific information about the specific REMS medication, and other new developments in the healthcare field. The field team leader 78 could provide clinician 56 with a single point-of-contact for all patients services and community care coordination. Following the instructions of the clinician 56, field team leader 78 could also coordinate team meetings for the patient 52 and in-home patient examinations.

Medical Support: Care management system 92 could have a medical support division 70 to help with various ancillary operations of a clinical practice. For clinical staff, care management system 92 may assist with staffing needs, training, or recruiting. Care management system 92 could also assist with administrative/clerical support or reception/customer service for the patient or family. Other medical support services that could be provided include document processing, health information management, medical transcription, medical services coding, etc.

Patient & Family Service Center: Care management system 92 could also have a patient service center 82 to provide other services to assist the patient 52 and family with other medical-related needs. For example, patient service center 82 may interact with third-party providers of financial assistance 80 to help patient 52 pay for medical expenses. Patient service center 82 may help in identifying suitable financial assistance programs or credit-granting companies.

Long-Term Care Coordinator: Care management system 92 could have a long-term care coordinator who performs casework duties and case management services for patients, families, and caregivers. The care coordinator could have a variety of duties, such as acting as a liaison between patients and the care facility, assist patients and their families with personal and environmental difficulties associated with the medical condition, ensure that the care facility staff are educated about the medication and its side effects, ensure that the patient meets all institutional criteria, and ensure product availability on the institution's formulary list.

Team Leader: Care management system 92 could have a team leader to manage the different personnel. Prescriber designee 94 could take on this role as the team leader. For example, long-term care coordinator 96, visiting nurse 98, field team leader 78, and field representative 74 could report directly to prescriber designee 94 to give status updates relevant to each of their respective case roles.

Final Remarks: The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof. Although this invention disclosure describes the present state of the FDA's regulations relating to the implementation of the “Clozapine Risk Evaluation and Mitigation Strategy”, this invention is intended to encompass any future regulatory changes or adjustments in the implementation thereof.

1. A method of managing the care of a patient who is on a REMS-required medication, comprising:
making a treatment plan for the patient, wherein the treatment plan comprises (i) a prescription for a medication for the patient, wherein the medication is regulated by a REMS program that has a REMS database; and (ii) a safety monitoring plan for blood testing from collection of blood samples taken from the patient at intermittent times,
transmitting the prescription to a pharmacy;
taking a blood sample from the patient;
analyzing the blood sample on a point-of-care testing equipment or at a clinical laboratory to generate a blood test result, wherein the blood test result includes the collection date of the blood sample;
reviewing the blood test result for one or more acceptable parameters;
based on the blood test result, dispensing the medication to the patient;
as the patient continues use of the medication, taking repeat blood samples from the patient;
based on the repeat blood test results, continuing to dispense the medication to the patient;
Wherein the improvement comprises:
having a monitoring hub that has personnel who is enrolled in the REMS program as a prescriber designee on behalf of the clinician;
wherein the monitoring hub receives from the clinician, patient information about the patient and the treatment plan for the patient;
wherein the monitoring hub receives the blood test results from the laboratory and enters the blood test results into the REMS database;
wherein the prescriber designee at the monitoring hub reviews the blood test results for acceptable parameter(s);
wherein, if a blood test result is not within acceptable parameter(s), the prescriber designee requests instructions from the clinician about changes to the treatment plan.

2. The method of claim 1, wherein the patient has Parkinson disease.

3. The method of claim 2, wherein the patient further has psychosis that is treatable with an antipsychotic medication.

4. The method of claim 3, wherein the REMS-required antipsychotic medication is clozapine.

5. The method of claim 4, wherein the blood test is for absolute neutrophil count (ANC).

6. The method of claim 5, wherein an acceptable parameter for the blood test is ANC 1500/μL.

7. The method of claim 1, wherein the monitoring hub further performs the task of enrolling the patient into the REMS program.

8. The method of claim 1, wherein the monitoring hub further performs the task of arranging for the blood collection to be performed on the patient.

9. The method of claim 8, wherein the monitoring hub has a visiting patient worker, wherein the visiting patient worker travels to the patient's residence and performs a blood draw on the patient to collect a blood sample.

10. The method of claim 9, wherein the visiting patient worker further transports the blood sample to the laboratory, or wherein the visiting patient worker further performs on-site blood testing to generate a blood test result and transmits the blood test result to the clinical laboratory or monitoring hub.

11. The method of claim 6, wherein the patient's ANC≥1500/μl for a duration of at least 6 months, and the prescriber designee at the monitoring hub changes the safety monitoring plan to reduce the frequency of blood collection from the patient.

12. The method of claim 11, wherein the prescriber designee changes the frequency of blood collection to about biweekly testing.

13. The method of claim 12, wherein the patient's ANC≥1500/μL for a duration of at least another 6 months, and the prescriber designee at the monitoring hub changes the safety monitoring plan to further reduce the frequency of blood collection from the patient.

14. The method of claim 13, wherein the prescriber designee changes the frequency of blood collection to about monthly testing.

15. A system for monitoring a patient on a REMS-required medication, comprising:
(a) a clinical practice comprising a clinician;
(b) a pharmacy comprising a pharmacist;
(c) a clinical laboratory;
wherein the clinician makes a treatment plan for the patient, wherein the treatment plan comprises (i) a prescription for a medication for the patient, wherein the medication is regulated by a REMS program that has a REMS database; and (ii) a safety monitoring plan for blood testing from collection of blood samples taken from the patient at intermittent times;
wherein the clinician transmits the prescription to the pharmacy;
wherein the clinical laboratory analyzes a blood sample taken from the patient to generate a blood test result, wherein the blood test result includes the collection date of the blood sample;
wherein the pharmacy reviews the blood test result for one or more acceptable parameters;
based on the blood test result, the pharmacy dispenses the medication to the patient;
as the patient continues use of the medication, the clinical laboratory analyzes further blood samples taken from the patient to generate further blood test results;
based on the further blood test results, the pharmacy continues to dispense the medication to the patient;
Wherein the improvement comprises:
(d) a monitoring hub that has personnel who is enrolled in the REMS program as a prescriber designee on behalf of the clinician;
wherein the monitoring hub receives from the clinician, patient information about the patient and the treatment plan for the patient;
wherein the monitoring hub receives the blood test results from the laboratory and enters the blood test results into the REMS database;
wherein the prescriber designee reviews the blood test results for acceptable parameter(s);
wherein, if a blood test result is not within acceptable parameter(s), the prescriber designee requests instructions from the clinician about changes to the treatment plan.

16. The system of claim 15, wherein the monitoring hub further performs the task of enrolling the patient into the REMS program.

17. The system of claim 15, wherein the monitoring hub further performs the task of arranging for the blood collection to be performed on the patient.

18. The system of claim 17, wherein the monitoring hub has a visiting patient worker, wherein the visiting patient worker travels to the patient's residence and performs a blood draw on the patient to collect a blood sample.

19. The system of claim 18, wherein the visiting patient worker further transports the blood sample to the laboratory.

20. The system of claim 15, wherein the REMS-required antipsychotic medication is clozapine.
